(19) United States
(12) Patent Application Publication
(10) Pub. No.: US 2026/0064810 A1
(43) Pub. Date: Mar. 5, 2026

(54) MATCHING DATA ITEMS IN LOWER-DIMENSIONAL SPACE USING GEOMETRY

(71) Applicant: Microsoft Technology Licensing, LLC, Redmond, WA (US)

(72) Inventors: Melanie Natividad PRADIER FERNANDEZ, Cambridge (GB); Javier GONZALEZ HERNANDEZ, Cambridge (GB)

(21) Appl. No.: 19/064,672

(22) Filed: Feb. 26, 2025

Related U.S. Application Data
(60) Provisional application No. 63/691,297, filed on Sep. 5, 2024.

(51) Int. Cl. G06F 18/22; G16H 10/60; G16H 50/70

(52) U.S. Cl. CPC G06F 18/22; G16H 10/60; G16H 50/70

(57) ABSTRACT

A computer-implemented method includes receiving input data items, each input data item comprising: first attributes representative of characteristics of the input data item, a treatment variable associated with the data item and an outcome variable representative of an outcome associated with the input data item. Second attributes of the input data items are generated from the first attributes, the second plurality of attributes having smaller dimensions than the first attributes. A first input data item having a first value for the treatment variable is selected; and a matching second input data item is selected based on a distance along a manifold between the first input data item and the second input data item, the second input data item having a second value for the treatment variable. The method provides a means of estimating the treatment effect of the treatment.

### CROSS-REFERENCE TO RELATED APPLICATION

This application claims priority to U.S. Provisional Patent Application No. 63/691,297, entitled “MATCHING DATA ITEMS IN LOWER-DIMENSIONAL SPACE USING GEOMETRY,” filed on Sep. 5, 2024, the disclosure of which is incorporated herein by reference in its entirety.

### BACKGROUND

In a wide range of circumstances and fields, it is necessary to estimate the effect of a treatment. This may be a treatment in the sense of a medical treatment or invention, but herein treatment refers broadly to any relevant action or intervention where it is desirable to estimate the effect of the treatment.

One existing means of estimating treatment effects is a randomized control trial (RCT), in which a population is randomly divided into a treatment population and a control population. The treatment is applied to the treatment population, but not the control population, which facilitates an analysis of the effect of the treatment. However, RCTs are expensive, time-consuming and can in some circumstances be unfeasible or unethical to run.

### SUMMARY

According to a first aspect of the disclosure, there is provided a computer-implemented method comprising: receiving a plurality of input data items, each input data item of the plurality of input data items comprising: a first plurality of attributes representative of characteristics of the input data item, the first plurality of attributes having a first plurality of dimensions, a treatment variable associated with the input data item and an outcome variable representative of an outcome associated with the input data item; generating, for each of the plurality of input data items, a second plurality of attributes from the first plurality of the second plurality of attributes having a second plurality of dimensions smaller than the first plurality of dimensions; selecting a first input data item having a first value for the treatment variable; determining, in respect of the first input data item, a matching second input data item based on a distance along a manifold between the second plurality of attributes of the first input data item and the second plurality of attributes of the second input data item in the representation space, the second input data item having a second value for the treatment variable, and estimating an effect of the treatment based the respective outcome variables of the first input data item and second input data item.

According to another aspect of the disclosure, there is provided a computer system comprising a processor and memory, the memory storing instructions which when executed by the processor cause the processor to execute the method of the first aspect.

According to another aspect of the disclosure, there is provided a non-transitory computer-readable medium storing instructions which when executed by a processor cause the processor to execute the method of the first aspect.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

### BRIEF DESCRIPTION OF THE DRAWINGS

To assist understanding of the present disclosure and to show how embodiments may be put into effect, reference is made by way of example to the accompanying drawings in which:

FIG. 1 is a schematic illustration of matching in Euclidean input space.

FIG. 2 is a schematic illustration of an example process of mapping input data items to a manifold and matching thereon.

FIG. 3 is a schematic block diagram illustrating an example process of learning a mapping between input space and the manifold.

FIG. 4 is a schematic flowchart of an example method.

FIG. 5(a)-(e) are experimental results based on a swissroll data set.

FIG. 6(a)-(d) are experimental results based on a motion capture data set.

FIG. 7 is a schematic block diagram of an example system for estimating treatment effects in the medical domain.

FIG. 8 is a schematic block diagram of an example system for estimating treatment effects using image data.

FIG. 9 is a schematic block diagram of an example retrieval augmented generation system.

FIG. 10 is a block diagram of an example computing system.

### DETAILED DESCRIPTION

Causal inference aims to estimate the effect of a treatment T on a given outcome Y in the presence of pre-treatment covariates X, which are also referred to as confounders based on observational data. As such, it avoids the cost and complexity involved in RCTs discussed above.

For example, in the medical domain the treatment may be a medical intervention (e.g. a particular drug), the outcome may be the survival rate, and the confounders may be attributes of the patient (e.g. age, weight, pre-existing conditions etc), each attribute having a corresponding value. In other domains, a treatment may be showing a specific kind of advertisement online versus not, a policy such as raising the national minimum wage of a country, or the introduction of a new policy in a company. However, as further discussed herein, the term “treatment” can broadly refer to a wide range of actions or interventions in various fields.

Confounders are responsible for the so-called confounding bias, an issue in observational studies where there exists an imbalance in the distributions of the treated and control units, due to the treatment not being randomly assigned to the observed population. The “units” in this context are individual members of the respective treatment and control populations.

One means of alleviating confounding bias is matching, where the goal is to pair suitable control units to each treatment unit (and vice versa) based on the similarity of their covariate information. In other words, matching aims to find, in respect of a treated unit, a control unit with similar confounders (e.g. another patient with similar attributes). From this matched pair, an estimate of the treatment effect can be determined as discussed below.

Traditional matching methods suffer from at least two limitations.

Firstly, they operate in the space of the raw input covariates, this input space hereinafter referred to as X, which is noisy and high-dimensional, so it becomes harder to find matches. For example, matching two patients that have hundreds or thousands of attributes is increasingly difficult. This problem increases as dimensionality increases, with distances becoming increasingly meaningless for higher dimensions. Secondly, observations are assumed to exist in Euclidean space.

The present disclosure assumes that the high-dimensional confounders lie on a manifold of much smaller dimensionality. This is the so-called manifold hypothesis, which is based on the assumption that the underlying structure of the manifold arises from causal mechanisms that induce statistical correlations among the confounders in a constrained manner.

For example, a sports trainer might want to estimate the effect of a particular training strategy (i.e. a treatment T) on an athlete's performance, the athlete being a treatment unit and their performance being an outcome Y. To estimate such an effect using observational data, the trainer might need to adjust for a wide variety of confounders, such as height, weight and other physiological measurements. Here, the similarity of two athletes should vary according to the natural differences in their physiology. Accordingly, a measure of distance between two athletes should respect the manifold structure-here, encoding the anatomical constraints-such that confounding bias in treatment effect estimation can be reduced.

In overview, the techniques discussed herein provide a means of estimating treatment effects by mapping input data items from an input space to a lower dimensional representation space having the form of a manifold. A match is then determined between a first input data item (e.g. a treated unit) and a second input data item (e.g. a control unit), based on a distance between the first input data item and the second input data item along the manifold in the representation space.

There now follows a further description of various concepts in matching treatment and control units.

Herein X denotes input (i.e. pretreatment) covariates, where X∈X. In other words, the input variables are in the input space X. There will be a plurality of input covariates for a given data item, such that the covariates effectively form a feature vector for the data item. T is a treatment variable T∈{0,1} associated with the data item. In other words, the treatment variable represents whether a data item (or the entity that the data item represents) has been treated, and thus whether it belongs to the treatment or control population, with 0 indicating that the data item is in the control population and 1 indicating that the data item is in the treatment population. Y is the outcome variable Y∈y associated with the data item. In other words, the outcome variable is some metric expressing the outcome of the application of the treatment (or not in the case of control units) to the data item (or the entity that the data item represents).

The assumption is that each observation (i.e. each data item—the terms are used interchangeably herein) has two potential outcomes Y(0) and Y(1), of which only one is observed. In other words, the potential outcomes differ depending on the treatment applied, but a single observation is either treated or not treated and so only one of the two outcomes is apparent. In formal notation, this can be expressed as Y=(1−T)Y(0)+TY(1), which is the outcome corresponding to the administered treatment T.

The main goal of matching is to infer the effect of the treatment by comparing the treated outcome Y_{*}(1) to that of a matched unit

$${\hat{Y}}_{*}^{match} ,$$

which plays the role of a hypothetical “twin” of the treated unit had they not been exposed to the treatment. In other words,

$${\hat{Y}}_{*}^{match}$$

aims to be as close as possible to the unobserved potential outcome Y_{*}(0). Herein the index “*” refers to a unit of interest—i.e. a subject unit.

In some examples, the matched unit is constructed as an average of the outcomes of a plurality of control units. However, in the following description the examples are explained with reference to nearest neighbour matching—i.e. the case where a single control unit is assigned to the treated unit of interest.

Let j^{†} denote the index of the nearest neighbour. A matched unit can then be constructed as:

$${\hat{Y}}_{*}^{match} = {\sum }_{j : {T}_{j} = 0} {w}_{j} {Y}_{j} , where \{ \begin{matrix}{w}_{j} = 1 & if j = {j}^{†} \\ 0 & if j \neq {j}^{†}\end{matrix}$$

In other words, the matched unit is constructed by setting the weight w of the outcome to 1 if the index corresponds to nearest neighbour and 0 otherwise.

To find the best match covariate match Xⱼ_{^{†}} for a treatment unit X_{*}, examples of the disclosure perform a nearest neighbour search among all control units according a distance metric d. For example, the search may be notated as:

$${j}^{†} = {arg min}_{j ∈ [ m ]} d ( {X}_{*} , {X}_{{j}^{†}} )$$

where m represents the set of possible indices of the control units. In other words, the matched unit is that which has the smallest distance from the treatment unit X_{*}, according to the distance metric d.

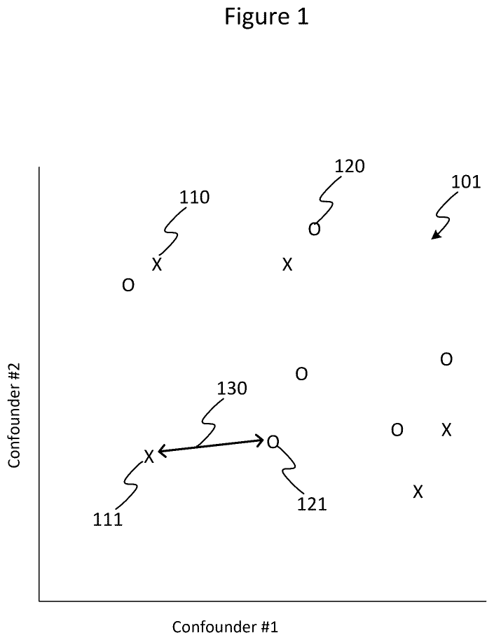

FIG. 1 provides a general illustration of these concepts, in a simplified form. In FIG. 1, the input space X 101 is two-dimensional for the sake of illustration, with the axes representing confounders. A plurality of data items are illustrated in the input space with treatment units 110 indicated by “x”s and control units 120 indicated by “o”s. Only one of each treatment unit 110 and control unit 120 is labelled for clarity. For a particular treatment unit 111, the nearest neighbour search identifies the closest matching control unit 121, as indicated by the arrow 130.

In the example of FIG. 1, the arrow 130 shows the distance in the input space X (i.e. the Euclidean distance). However, in examples of the disclosure, the distance is calculated in a different representational space—the manifold—as will now be discussed in detail.

Examples of the disclosure assume the manifold hypothesis discussed above. This hypothesis states that the high-dimensional confounders X lie near a low-dimensional and non-linear manifold embedded in the input space. The low-dimensional structure arises due to constraints from e.g. physical laws and nature or other relevant relationships that govern the distribution of the input data items. In other words, the geometry of the manifold results from the underlying causal mechanisms between the different confounders.

The manifold hypothesis is satisfied in a wide range of fields. An example (but by no means exhaustive) set of data types where it is fulfilled include image vectors representing 3D objects under different angles and light conditions, phonemes in speech signals, or video streams.

In examples, the treatment effect varies smoothly along the manifold of the confounders. For example, in the aforementioned example of sports trainers, it would be expected that athletes with similar anatomical constraints (reflected by the manifold structure) would respond similarly to different training strategies. As another example, a chemist might want to assess the effect of adding a catalyst to a chemical reaction (i.e. a treatment) on the chemical reaction rate (i.e. an outcome) given experimental conditions such as pH, purity of reactants, pressure, temperature, etc (i.e. the confounders). As we move along the space of possible experimental conditions (i.e. the manifold), a smooth variation of the treatment effect is expected.

Examples herein therefore use the distance between treated and control units along the manifold as the distance metric d.

There now follows a brief discussion of manifolds, and Riemannian manifolds in particular. The following definitions may apply to examples of the disclosure.

A d-dimensional smooth manifold M is a topological space which locally resembles the Euclidean space [symbol] ^{d} and has a smooth structure.

A Riemannian manifold is a differentiable (and thus smooth) manifold M provided with a Riemannian metric tensor G.

A Riemannian metric G on a manifold M is a smooth function that assigns a symmetric positive definite matrix to any point z∈M. A Riemannian metric defines at each point z a smoothly varying inner product in the tangent space T_{z}M. As the name suggests, the tangent space is a generalization of tangent lines in two-dimensional space to the higher-dimensional space of the manifold. The inner product is defined as:

$${〈 {z}_{1} , {z}_{2} 〉}_{z} = {z}_{1}^{T} G ( z ) {z}_{2}$$

Where z₁, z₂∈T_{z}M and z∈M. Intuitively, a Riemannian metric G defines an infinitesimal notion of distance on the manifold M. That is to say, the metric provides a means of measuring infinitesimally small distances on the smooth manifold, such that the length of a smooth curve along the manifold can be determined by integrating the infinitely small distances along the curve.

More formally, the length of a smooth curve γ: [0, 1]→ [symbol] can be defined as:

$$Length ( γ : G ) = {\int }_{0}^{1} \sqrt{{{γ}^{′} ( r )}^{T} G ( γ ( r ) ) {γ}^{′} ( r ) dr}$$

Where

$${γ}^{′} ( r ) = \frac{d}{d r} γ ( r )$$

denotes the velocity of the curve. The distance between two points on the manifold M is defined as the shortest curve connecting them.

Consequently, in examples, the distance metric d discussed above makes use of this length along the manifold. Consequently, the distance metric d is referred to herein as a Riemannian distance:

$${d}_{ℛ} ( {X}_{*} , {X}_{j} ) = {min}_{Y j} Length ( {γ}_{j} , G )$$

This is also known as the geodesic curve—the shortest curve connecting the two points along the manifold. γⱼ: [0, 1]→Z is a curve representing the latent representations of the confounders. That is to say γⱼ(0)=Z_{*} γⱼ(1)=Zⱼ. Consequently, [symbol] may also be referred to as a geodesic distance.

In one example, to compute the geodesic distances, a parameterized (i.e. discretized) geodesic curve is instantiated, and its parameters are optimised by minimizing the following second-order ordinary differential equation:

$${γ}^{″} = - \frac{1}{2} {{G}^{- 1} [ \frac{δ vec G}{δ γ} ]}^{T} ( γ ⊗ {γ}^{′} )$$

where vec G stacks the columns of the matrix G and ⊗ denotes the Kronecker product.

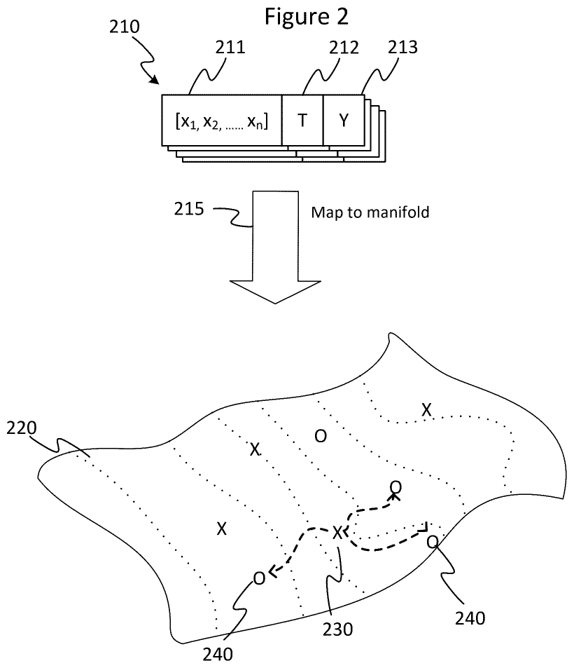

FIG. 2 illustrates the process of mapping input data items to the manifold and calculating the distance [symbol] . A plurality of input data items 210 are received, each comprising a plurality of confounders 211 in the form of a feature vector, as well as a treatment variable 212 and outcome variable 213. The number n of confounders 211 corresponds to number of dimensions of the input space X.

As generally indicated by the arrow 215, the confounders 211 of the input data items 210 are mapped into the lower-dimensional space of the manifold 220. It will be appreciated that the manifold is shown as a 3-dimensional surface, but in reality the manifold 220 is likely to have many more dimensions. The latent space in which the manifold 220 exists is referred to as Z herein. The process of learning the mapping between the input space X and the manifold 220 is discussed in more detail later.

As in FIG. 1, data items in the manifold space corresponding to treatment units 230 are represented by “x”s and data items corresponding to control units 240 are represented by “o”s.

As indicated by dashed arrows, the distance between a treatment unit 231 and the control units 240 is a shortest distance along the surface of the manifold. That is to say, the distance follows the contours of the manifold—i.e. it is the geodesic curve. Consequently, the nearest neighbour—i.e. the matched unit—is the control unit 240 with the minimum of the geodesic curves between the treatment unit 231 and the control units 240.

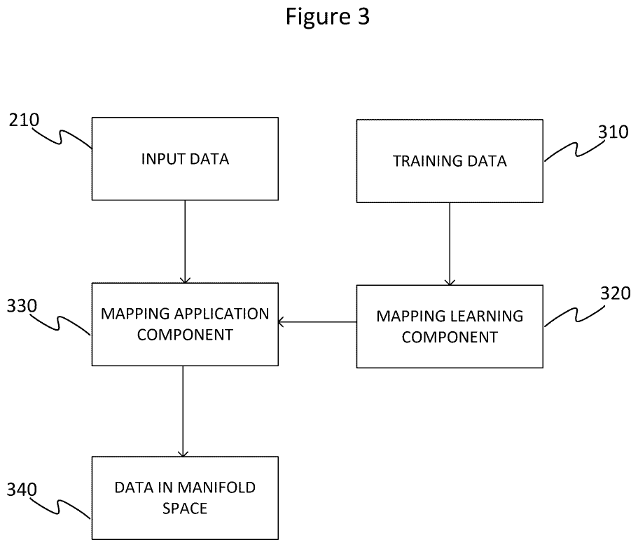

Referring to FIG. 3, there now follows a discussion of the process of learning the mapping between the input space and the latent space of the manifold. It will be understood that learning the topology of the manifold, learning the manifold or latent space, learning the Riemannian metric and learning the mapping between the input space and the latent space all effectively amount to the same task. That is to say, in learning the topology of the manifold, which is represented by the Riemannian metric G, it necessarily follows that the mapping between the input space and the manifold is determined. Accordingly, further references to these tasks can be considered to be interchangeable herein.

In general, the process involves using a set of data items 310 to learn the latent space. For convenience, these data items are referred to as training data items, though as will be apparent from the discussion that follows, the techniques involved may not involve training in the traditional supervised machine learning sense.

In some examples, the training data 310 is a held-out portion of the input data items 210. For example, 10% or 20% or any other suitable amount of an original set of the input data items 210 is designated as the training data 310. However, in other examples, the training data 310 is from a different data set than to the input data.

The training data 310 then forms input to a mapping learning component 320. A number of options are envisaged for the mapping learning component 320.

In a first example, a linear projection is determined between the input space and the latent space based on the training data 310. For example, Principal Components Analysis (PCA) may be employed to determine a suitable linear projection based on the training data 310. In this example, a parameterized Riemannian metric is fitted to the output of the linear projection—i.e. it is fitted to the latent representation. For example, a parametric Local Inverse Variance (LIV) Riemannian metric is fitted in the latent space Z. The LIV metric may be defined as the inverse of a local diagonal covariance matrix with entries:

$${G}_{j j} ( z ) = {( {\sum }_{i = 1}^{N} {w}_{i} ( z ) {( {z}_{i j} - {z}_{j} )}^{2} + ρ )}^{- 1} where {w}_{i} ( x ) = exp ( - \frac{{‖ {z}_{i} - z ‖}^{2}}{2 {σ}^{2}} )$$

In the above, zᵢⱼ is the j^{th} dimension of the i^{th} observation in the training data 310. The parameter σ controls how fast uncertainty increases as points move away from the manifold. ρ is needed for numerical stability, and influences the magnitude that the metric values can take. Effectively, the LIV assumes a locally Euclidean metric, which is then regularized to have a large volume measure in regions of the feature space far away from observations. The notion underlying the metric is that shortest paths on the data manifold should remain close to the observed data, and curves should avoid crossing low-density (and thus high-uncertainty) regions of the manifold. A key advantage of this metric is that geodesic computation is less costly, as off-diagonal terms in G are zero. Furthermore, the PCA approach is generally relatively computationally efficient, but at the expense of not being guaranteed to preserve geometric structure in detail.

In this example “fitting” the metric may involve experimenting with different values of σ and/or ρ based on the training set, to arrive at a suitable value for each parameter.

In a second example, a probabilistic latent variable model is trained. This model is trained to jointly learn Z and [symbol] [G] (i.e. an estimate or expected G). The model may be trained to propagate Jacobian information.

Various other example techniques may be adopted for learning the mapping and Riemannian metric G. These include Isomap, diffusion models, gaussian process latent variable models, kernel PCA techniques, multidimensional scaling and so on. It will be understood that techniques for non-linear dimensionality reduction—i.e. manifold learning—are well established and any suitable technique may be applied to learn the manifold.

Once the mapping to the manifold has been learned, this mapping is applied to the input data 210, as represented by mapping application component 330 in FIG. 3. This may involve iteratively applying the mapping to each of the feature vectors (i.e. plurality of confounders) 211 of the input data 210. This results in data item corresponding to each input data item in the latent space of the manifold, represented by block 340 in FIG. 3. In other words, the mapping process results in a manifold space feature vector for each input feature vector, wherein the manifold space feature vector is representative of a location in the manifold space. The feature vector in the manifold space is of lower dimensionality than the feature vector in the input space, on account of the manifold space having fewer dimensions than the input space. In examples where a trained model is used, this involves providing the input confounders 211 to the model and receiving in response a feature vector in the manifold space. In other examples, it involves the metric fitted to the training data.

At this stage, matching can be carried out between a treated unit and a control unit. In more detail, returning to FIG. 2, an input data item represented in the manifold space is selected. The distance [symbol] discussed above can then be computed between the selected treated unit and each control unit represented in the manifold space. The control unit with the smallest [symbol] in respect of the treatment unit is selected as the match.

The matched pair can then be used to determine the treatment effect, by comparing the outcome variables thereof. Particularly, the estimated individual treatment effect (ITE) can be calculated as follows:

$$= {Y}_{*} - {\hat{Y}}_{*}^{match}$$

In some examples, a plurality of treatment units are matched to respective control units. This then allows the calculation of an average treatment effect (ATE), which as the name suggests takes an average over the individual treatment effect of each pair. In one example, this is calculated according to the following formula:

$$= \frac{1}{N} {\sum }_{i = 1}^{N} {( {Y}_{i} - {\hat{{Y}_{i}}}^{match} )}^{{T}_{i = 1}} {( {\hat{Y}}_{i}^{match} - {Y}_{i} )}^{{T}_{i = 0}}$$

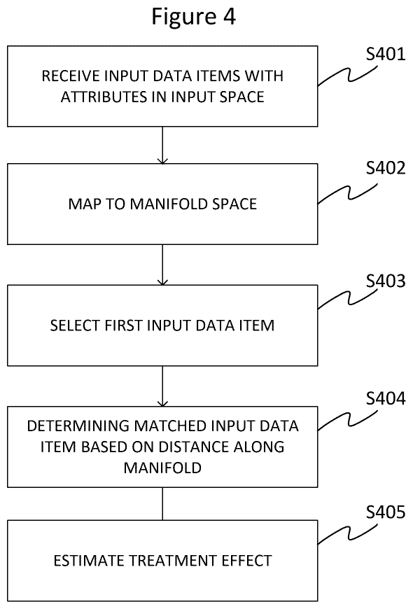

FIG. 4 illustrates a computer-implemented method according to examples of the disclosure.

In step S401, a plurality of input data items are received. Each input data item comprises a first plurality of attributes in an input space having a first plurality of dimensions. These attributes may form a feature vector as discussed above. Each input data item comprises a treatment variable representative of whether a treatment has been applied to the data item. Each input data item also comprises an outcome variable representative of an outcome associated with the input data item.

In step S402, the attributes of the input data items are mapped from the input space into a representation space having a second plurality of dimensions smaller than the first plurality of dimensions. In other words, a second plurality of attributes is generated from the first plurality of attributes, wherein the dimensionality of the plurality of second attributes are smaller than the plurality of first attributes. The representation space takes the form of a manifold, such as a Riemannian manifold as discussed herein.

In step S403, a first input data item having a first value for the treatment variable is selected. The first value may be representative of the treatment being applied, such that the first input data item is a treatment unit.

In step S404, a matching second input data item for the first input data item is determined. The second input data item has a second value for the treatment variable different from the first value. For example, the second input data item has a value that is representative of the treatment not being applied, such that the second input data item is a control unit. The determining is based on a distance along the manifold between the first input data item and the second item in the representation space.

In step S405, a treatment effect is estimated based on the respective outcome variables of the first and second input data items. However, as discussed in more detail below, in some examples the output of the matching may find utility without necessarily proceeding to estimating a treatment effect.

There now follows a discussion of experimental results associated with the techniques discussed above.

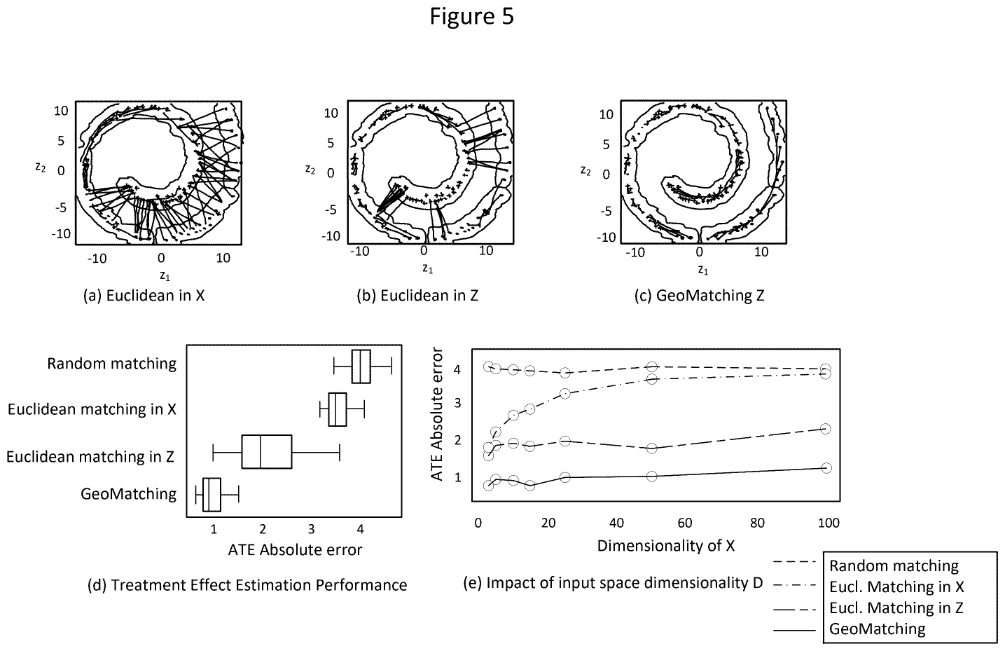

FIG. 5(a)-(e) illustrates a first set of experimental results that are based on the classic swissroll dataset. In the experiment, 200 observations (i.e. input data units) were generated where the covariates X follow a 3D-swiss roll manifold, the treatment variables Tare Bernoulli draws from a smoothly varying sigmoid function along the swissroll manifold, and outcome variables Y are drawn from linear models also along the swissroll manifold. To assess the impact of input dimensionality on the different matching strategies, the three-dimensional swissroll is embedded in a space of higher dimensionality—each of 3, 5, 10, 15, 25, 50 and 100 dimensions—by adding additional noisy input dimensions.

The input data units are projected into a 2D latent space. Both PCA and Isomap were experimented with, each returning in similar latent representations preserving the geometry of the swissroll.

FIG. 5(a) illustrates a first baseline approach, in which Euclidean distance is used for matching in the input space. FIG. 5(b) illustrates a second baseline approach, in which Euclidean distance is again used, but this time in the latent space Z. The techniques described above—referred to as GeoMatching—are shown in FIG. 5(c). It can be clearly seen that the present techniques successfully couple treatment units (referred to as “cases” in the figures) and control units along the manifold, whereas the baselines disregard the manifold geometry and cross areas of high uncertainty.

FIGS. 5(d) and 5(e) illustrate the absolute error of the ATE for the baselines and the present techniques, illustrating that the present techniques provide the lowest ATE Error.

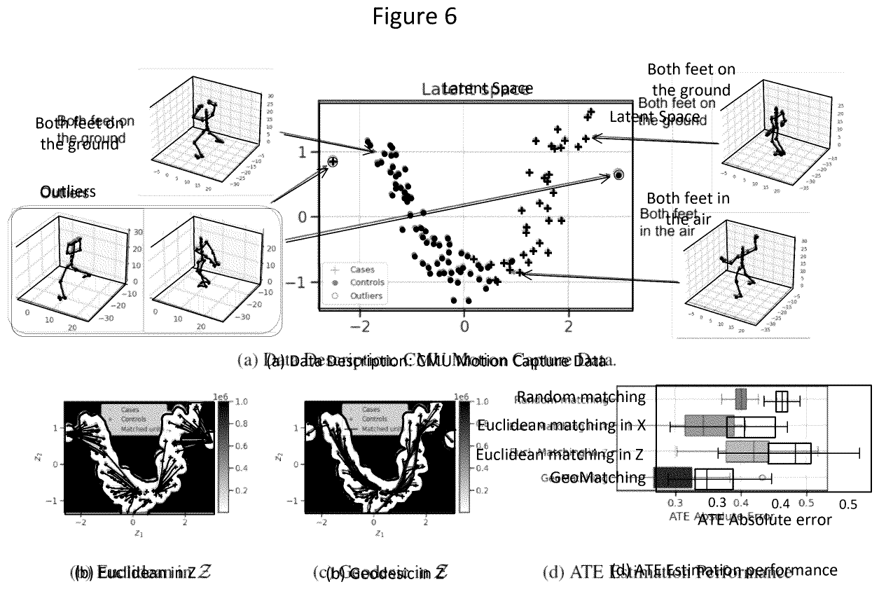

FIG. 6(a)-(d) illustrates a second set of experimental results, this time in human motion capture data. The data comprises of real-world input covariates from the captured motion, with synthetic treatment and outcome variables. This experiment illustrates the effectiveness of the present techniques in handling high-dimensional realistic covariates and the robustness of GeoMatching against outlier confounders, in contrast to the geometry-agnostic baselines.

Specifically, the input data is motion 16 from subject 22 from the CMU Motion Capture Database—http://mocap.cs.cmu.edu, which is a repetitive jumping jack motion. Each observation corresponds to a human pose acquired by a marker-based motion capture system.

The CMU motion capture data is adapted for the task of treatment effect estimation as follows. The subject of the motion capture is assumed to be playing Double Dutch—a jump rope game in which two people turn two long ropes in opposite directions while multiple players jump simultaneously, entering the rope area one at a time. X is the motion capture of the current player jumping. T is the strategy of entrance by the player to the rope area, which is either slow (T=0) or fast (T=1). Y is the probability of a successful entrance, which is when the ropes do not trip the new jumper's feet.

To make the data more challenging, some time frames of the data set were intentionally perturbed to simulate outlier confounders in the data set—i.e. players jumping in a weird, unnatural manner or motion capture sensors being defective.

FIG. 6(a) is a visualization of the data in a 2D latent space using the PCA technique discussed above, which preserves the geometric/periodic structure of the data. FIGS. 6(b) and 6(c) respectively illustrate the baseline performance of Euclidean matching in Z and the performance of present techniques. It can be seen that the baseline is sensitive to the outliers, matching many units across the uncertain region outside the manifold, whereas the present techniques are more robust against the outliers. FIG. 6(d) illustrates ATE absolute error in a similar manner to FIG. 5(d), again illustrating the superior performance of the present techniques.

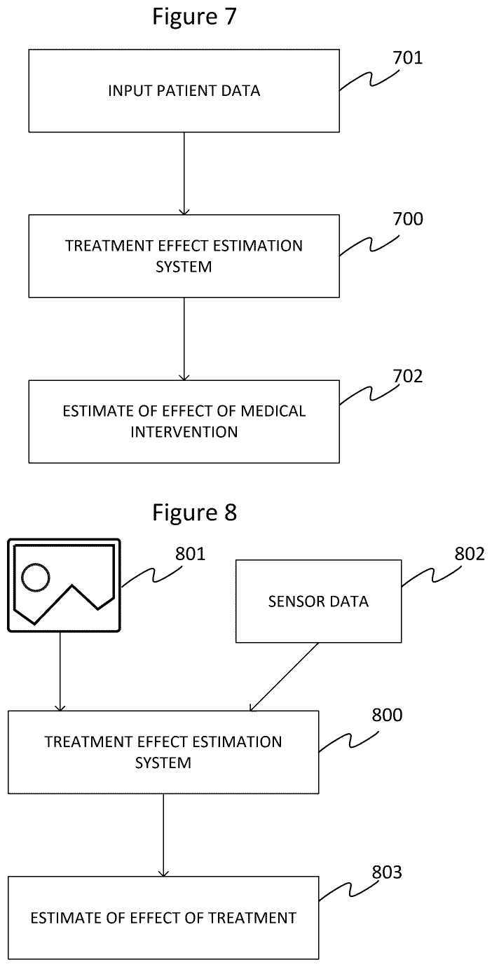

It will be appreciated that the techniques discussed above have a wide range of applications. A selection of these applications will now be discussed with respect to FIGS. 7-10.

FIG. 7 illustrates a first example system 700 for estimating treatment effects in the medical domain. The system 700 is a computer system configured to execute some or all of the techniques discussed above.

In the example, the input data 701 is patient data. The confounders X may express attributes of each patient, such as height, weight, age, presence of existing conditions and the like. The confounders may for example be in tabular form (i.e. forming a feature vector).

The treatment variable T represents a particular medical intervention, such as a drug or other therapy. The outcome variable Y represents some measure of the patient's health, such as a survival rate. The output 702 of the system 700 is an estimate of the treatment effect of the medical intervention. In other words, it is a medical outcome of the patient, such as a survival rate. Consequently, the system 700 is able to estimate the efficacy of the medical intervention.

FIG. 8 illustrates a second example system 800 for estimating treatment effects using image data. The system 800 is a computer system configured to execute some or all of the techniques discussed above, and output an estimate 803 of the effect of a treatment.

In the example, the input data 801 is image data. The confounders may represent the image—for example being pixel values—or may be data derived from the image, such as motion capture data, pose estimations, object detections and so on.

In one example, the image data 801 comprises medical images such as X-ray images, CT scans, MRI scans or the like. The treatment variable may again in this example represent a medical intervention and the outcome variable may represent a medical outcome.

However, image data 801 may be used in a wide variety of other fields for estimating treatment effects. For example, the image data 801 may be agricultural images, e.g. of plant products such as crops. The image data 801 may be reflective of the plant products condition thereof. In such an example, the treatment may be a pesticide or other policy, condition or intervention applied in an agricultural setting. The outcome may be some metric of crop yield or quality or the like.

In other examples, the image data 801 may pertain to industrial machinery, with the treatment reflecting a maintenance regime, modification or other policy or intervention applied to the industrial machinery. In such examples, the outcome variable may be a quality of the output of the machinery (e.g. number of items failing quality control), an uptime of the machinery or any other suitable metric codifying some aspect of the performance of the machine.

In addition to, or instead of, image data 801, the input data may be suitable sensor data 802 reflecting e.g. the state of plant products, the state of industrial machinery, and so on.

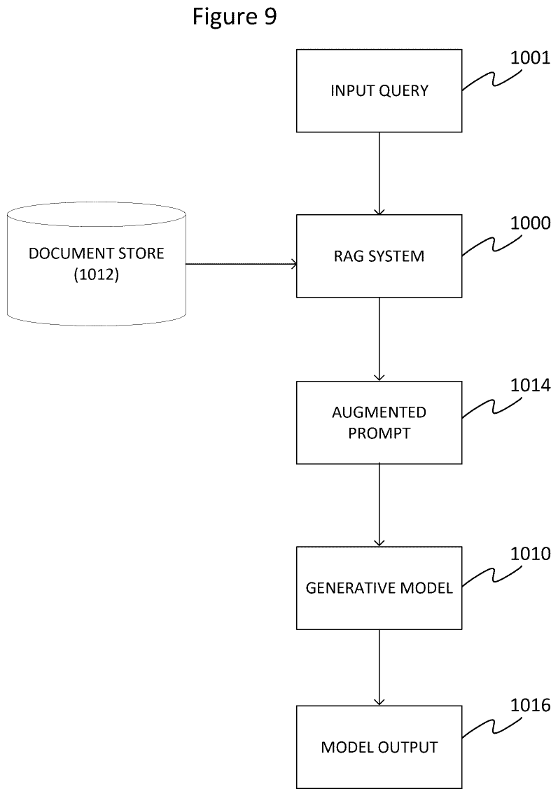

FIG. 9 illustrates another application of the example techniques. In this example, the system 1000 is a retrieval augmented generation (RAG) system for a generative model 1010. The generative model 1010 is configured to receive an input, which may be in the form of a textual prompt, and in response generate an output. An example generative model is a large language model (LLM) such as one of the GPT models provided by Open AI®, which is configured to provide output in the form of text. Equally, the generative model may be an image generation model (e.g. a diffusion model such as DALL-E, also provided by Open AI®), a video generation model, an audio generation model or any suitable multi-modal model that receives inputs and/or outputs of different modalities.

RAG is a mechanism for augmenting the input to the generative model 1010, by including documents (represented by document store 1012), in the input. To this end, an input query 1001 is received. In some examples, this is a user query input by a user via a suitable user interface, though in other examples the input query 1001 may be a metaprompt or other stored template query. The term query is not intended to reflect that the input query need always include a question—it encompasses instructions, statements or any other input that causes the generative model 1010 to provide a response. The input query may therefore also be referred to as an input. The RAG system 1000 is configured to retrieve one or more relevant documents from document store 1012, based on the input query 1001 and include them in an augmented input 1014. This augmented input (e.g. an augmented prompt) 1014 is then input to the generative model 1010, which provides a suitable output 1016.

These techniques are intended to provide improved output from the generative model 1010 compared to merely inputting the input query 1001 into the generative model 1010, in that the relevant knowledge or data in the documents of the document store 1012 provides further useful context to the generative model 1010.

In some background techniques, the retrieval of the documents is based on embedding the query and documents in an embedding space, and using a distance metric (e.g. cosine distance) to retrieve the most relevant document(s) from the document store. In contrast, system 1000 makes use of the techniques described herein to retrieve relevant documents from the document store 1012.

Accordingly, in this example, the input query 1001 is the first input data item, and the documents in the document store 1012 are potential options for the second input data item—i.e. they are candidates for matching. The attributes for each of the query 1001 and documents may be any suitable features encoding the content thereof. For example, the attributes maybe the tokens of the text of the input query and document respectively. The input query is mapped or embedded into a lower dimensional space, wherein the embedding is of a dimensionality that preserves the geometric structure (i.e. manifold). One or more matching documents are determined for the input query using the distance along the manifold. These documents are then included in the prompt.

Notably, in this example, the procedure stops at the matching stage, without progressing to estimate a treatment effect.

Documents in this context may be text documents, web pages, database entries, images and so on. In other words, this example applies broadly to retrieving results based on queries using the distance along the manifold.

Furthermore, whilst the example illustrated pertains to RAG, it will be appreciated that the techniques also apply to retrieving results based on queries in other settings. In other words, the generation of the augmented prompt 1014 including the retrieved document and input of the prompt 1014 into the generative model 1020 to provide an output 1016 is optional. Instead, system 1000 returns one or more matching documents.

Various alterations or modifications to the above-described examples are possible within the scope of the disclosure.

Whilst several techniques have been discussed for mapping from high dimensional input space to the lower dimensional space of the manifold, it will be understood that these examples are by no means exhaustive, and any suitable mapping technique may be applied.

Whilst the techniques discussed herein use a nearest neighbour approach to determine a matching control unit for a treatment unit, it will be understood that other approaches may be applied. For example, a plurality of proximate control units may be selected and averaged or otherwise combined to form a matched control unit. Equally, other suitable search techniques may be employed, for example so as to avoid calculating the distance between the treatment unit and every control unit in the data set.

Although the discussion above proceeds on the basis that a treatment unit is selected and a control unit is determined, the techniques may equally select a control unit and determine a matching treatment unit. The selection of the matching unit may in some circumstances employ selection without replacement, such that a matched unit cannot be again matched to another unit.

Furthermore, whilst the examples above involve a binary treatment variable, in other examples the treatment variable may take on more than two values. That is to say, the techniques herein apply to situations in which a plurality of treatments are possible and are applied to different ones of population represented by the input data. Still further, whilst the discussion above refers to control units, this need not imply that the control units are “untreated” in the sense of having no treatment or a placebo treatment applied-instead the control units may represent a different treatment. That is to say that a first value of the treatment variable may correspond to a first treatment, and a second value of the treatment variable may correspond to a second, different treatment.

Various other applications are envisaged, including in estimating athletic performance, assessing chemical reactions and so on.

More broadly, the techniques herein can be used in any situation when an action is performed on any form of causal system (the ‘target’ system) to purposively achieve a measurable technical effect in the causal system. The only requirement in this respect is that the technical effect is quantifiable and can be measured in respect of a performed action with sufficient accuracy and precision to achieve the required technical effect. Such measurements may be performed using any technical means/components/devices, such as sensors (e.g. one or more image sensors, audio/pressure sensors, temperature sensors, network sensors and/or electrical sensors and the like) software monitoring (e.g. resource monitoring components running as part of an operating system, or in firmware, network traffic monitoring software etc) on any form of technical system (physical or logical) exhibiting causal properties. By learning causal relationships within a causal system (i.e. by estimating the treatment effect associated with an action), the predicted technical effect of an action, enabling the action to be selected and performed with the aim of controlling that technical effect based on technical considerations concerning the causal properties of the target system in which the technical effect is sought to be achieved.

As further examples, in the manufacturing industry, causal inference can help quantitatively identify the impact of different factors that affect product quality, production efficiency, and machinery performance in manufacturing processes. By understanding causal relationships between these factors, manufacturers can optimize their processes, reduce waste, and improve overall efficiency. As another example, in the field of engineering, causal inference can be used for root cause analysis and identify underlying causes of faults and malfunctions in machines or electronic systems such as vehicles or unmanned drones (e.g. aircraft systems). By analyzing data from sensors, maintenance records, and incident reports, causal inference methods can help determine which factors are responsible for observed issues and guide targeted maintenance and repair actions. In genome-wide association studies (GWAS), causal inference may be used, for example, to associate between genetic variants and a trait or disease, accounting for potential confounding factors, which in turn may allow therapeutic treatments to be developed or refined.

In any of the examples discussed herein, the techniques may further involve further use of the estimated treatment effect. In some examples, the estimated treatment effect may be displayed to a user. In other examples, the estimated treatment effect may form the input to another technical process. For example, upon determining that the estimated treatment effect exceeds a threshold (and thus the treatment is effective), a computer system may take one or more actions. For example, upon determining that a maintenance regime is successfully in reducing machine downtime or increasing the quality of output, the same maintenance regime may be applied to other machines. Similarly, an agricultural machine may be controlled to apply treatment (e.g. fertilizer) to other areas after a determination that the treatment is effective. Analogously, similar actions may be automatically taken in respect of any of the applications discussed herein.

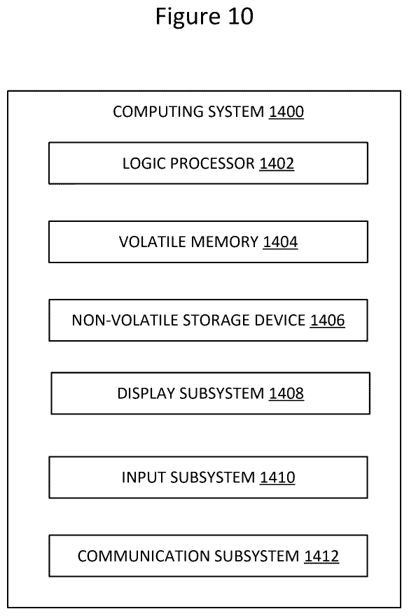

FIG. 10 schematically shows a non-limiting example of a computing system 1400 that can enact one or more of the methods and processes described above. Computing system 1400 is shown in simplified form. Computing system 1400 may embody any of the computer devices 700, 800, 900 or 1000 described above, or any other computer device discussed herein. Computing system 1400 may take the form of one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches and head mounted augmented reality devices.

Computing system 1400 includes a logic processor 1402, volatile memory 1404, and a non-volatile storage device 1406. Computing system 1400 optionally includes a display subsystem 1408, input subsystem 1410, communication subsystem 1412, and/or other components not shown in FIG. 10.

Logic processor 1402 includes one or more physical devices configured to execute instructions. For example, the logic processor is configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor includes one or more physical processors (hardware) configured to execute software instructions. Additionally or alternatively, the logic processor includes one or more hardware logic circuits or firmware devices configured to execute hardware-implemented logic or firmware instructions. Processors of the logic processor 1402 may be single-core or multi-core, and the instructions executed thereon may be configured for sequential, parallel, and/or distributed processing. Individual components of the logic processor optionally are distributed among two or more separate devices, which may be remotely located and/or configured for coordinated processing. In examples, aspects of the logic processor are virtualized and executed by remotely accessible, networked computing devices configured in a cloud-computing configuration. In such a case, these virtualized aspects are run on different physical logic processors of various different machines, it will be understood.

Non-volatile storage device 1406 includes one or more physical devices configured to hold instructions executable by the logic processors to implement the methods and processes described herein. When such methods and processes are implemented, the state of non-volatile storage device 1206 may be transformed—e.g., to hold different data.

Non-volatile storage device 1406 may include physical devices that are removable and/or built-in. Non-volatile storage device 1206 includes any of optical memory (e g., CD, DVD, HD-DVD, Blu-Ray Disc, etc), semiconductor memory (e g., ROM, EPROM, EEPROM, FLASH memory, etc.), and/or magnetic memory (e.g., hard-disk drive), or other mass storage device technology. Non volatile storage device 1406 includes any of nonvolatile, dynamic, static, read/write, read-only, sequential-access, location-addressable, file-addressable, and/or content-addressable devices. It will be appreciated that non-volatile storage device 1406 is configured to hold instructions even when power is cut to the non-volatile storage device 1406.

Volatile memory 1404 may include physical devices that include random access memory. Volatile memory 1404 is typically utilized by logic processor 1402 to temporarily store information during processing of software instructions. It will be appreciated that volatile memory 1404 typically does not continue to store instructions when power is cut to the volatile memory 1404.

Aspects of logic processor 1402, volatile memory 1404, and non-volatile storage device 1406 may be integrated together into one or more hardware-logic components. Such hardware-logic components include, for example, field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 1400 typically implemented in software by a processor to perform a particular function using portions of volatile memory, which function involves transformative processing that specially configures the processor to perform the function. Thus, a module, program, or engine can be instantiated via logic processor 1402 executing instructions held by non-volatile storage device 1406, using portions of volatile memory 1404. It will be understood that different modules, programs, and/or engines can be instantiated from the same application, service, code block, object, library, routine, API, function, etc. Likewise, the same module, program, and/or engine can be instantiated by different applications, services, code blocks, objects, routines, APIs, functions, etc. The terms “module,” “program,” and “engine” can encompass individual or groups of executable files, data files, libraries, drivers, scripts, database records, etc.

When included, display subsystem 1408 can be used to present a visual representation of data held by non-volatile storage device 1406. The visual representation takes the form of a graphical user interface (GUI). Because the herein described methods and processes change the data held by the non-volatile storage device, and thus transform the state of the non-volatile storage device, the state of display subsystem 1408 may likewise be transformed to visually represent changes in the underlying data. In examples, display subsystem 1408 includes one or more display devices utilizing virtually any type of technology. Such display devices can be combined with logic processor 1402, volatile memory 1404, and/or non-volatile storage device 1406 in a shared enclosure, or such display devices are peripheral display devices.

When included, input subsystem 1410 comprises or interfaces with one or more user-input devices such as a keyboard, mouse, touch screen, or game controller. In some examples, the input subsystem comprises or interfaces with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 1412 is configured to communicatively couple various computing devices described herein with each other, and with other devices. Communication subsystem 1412 may include wired and/or wireless communication devices compatible with one or more different communication protocols. As non-limiting examples, the communication subsystem is configured for communication via a wireless telephone network, or a wired or wireless local- or wide-area network. In some examples, the communication subsystem allows computing system 1400 to send and/or receive messages to and/or from other devices via a network such as the internet.

Additional example features of the disclosure are set out below.

According to a first aspect of the disclosure, there is provided a computer-implemented method comprising: receiving a plurality of input data items, each input data item of the plurality of input data items comprising: receiving a plurality of input data items, each input data item comprising: a first plurality of attributes representative of characteristics of the input data item, the first plurality of attributes having a first plurality of dimensions, and a treatment variable associated with the input data item; generating, for each of the plurality of input data items, a second plurality of attributes from the first plurality of attributes, the second plurality of attributes having a second plurality of dimensions smaller than the first plurality of dimensions; selecting a first input data item having a first value for the treatment variable; and determining, in respect of the first input data item, a matching second input data item based on a distance along a manifold between the second plurality of attributes of the first input data item and the second plurality of attributes of the second input data item, the second input data item having a second value for the treatment variable.

Each input data item may further comprise an outcome variable representative of an outcome associated with the input data item. The method may further comprise: estimating an effect of the treatment based the respective outcome variables of the first input data item and second input data item. The estimated effect may be an individual treatment effect.

The treatment variable being associated with the input data item may comprise the treatment variable being representative of whether a treatment has been applied to the data item. Being representative of whether a treatment has been applied to the data item may refer to the treatment being applied to an entity that the data item represents. Similarly, the outcome variable may be representative of an outcome associated with the entity that the data item represents.

The method may comprise selecting a plurality of further first input data items having the first value for the treatment variable. The method may comprise determining respective matching further second input data items for each of the plurality of further first input data items, the respective further second input data items having the second value for the treatment variable. The method may comprise estimating an average treatment effect based on the respective outcome variables of each further first input data item and its respective matched further second input data item.

The first plurality of attributes may be in an input space. The second plurality of attributes may be in a representation space.

The mapping may comprise determining, based on a training data set, a mapping between the input space and the representation space; applying the determined mapping to the plurality of attributes of the input data items.

Determining the mapping may comprise determining a linear projection from the input space to the representation space.

Determining the mapping comprises training a non-linear dimensionality reduction model based on the training data set. Applying the determined mapping comprises providing the plurality of attributes of the input data items to the model and in response receiving feature vectors representing the input data items in the embedding space.

Determining the mapping may comprise learning a manifold metric. The metric may be a Riemannian metric. The manifold may be a Riemannian manifold. The Riemannian metric may encode the geometry of the manifold. The distance along the manifold may be calculated using the manifold metric.

Determining the matching second input data item may comprise determining a nearest neighbour of the first input data item having the second value for the treatment variable.

Determining the matching second data item may comprise determining a plurality of matching second input data items based on the distance along the manifold; and generating a matched data item based on the plurality of proximate second input data items. In other words, the matching second data item may be constructed from the plurality of matching second input data items.

The distance along the manifold may be a geodesic curve.

The attributes may be representative of patient data. The treatment may be a medical intervention. The outcome variable may be representative of a medical outcome of a patient.

The attributes may be representative of image data.

The attributes may be representative of sensor data.

The attributes may be representative of plant products, such as crops. The treatment may be application of a pesticide or another agricultural intervention. The outcome variable may represent a crop yield or quality.

The attributes may be representative of industrial machinery. The treatment variable may reflect a maintenance regime applied to the industrial machinery. The outcome variable may reflect an uptime of the industrial machinery or a quality of output of the industrial machinery.

The first value of the treatment variable may correspond to a query. The second value of the treatment variable may correspond to potential results of the query. Determining the matching second input data item may comprise determining a result of the query. The attributes may be representative of documents, such that the query is a document query.

The method may comprise generating an input for a generative machine learning model including the query and the result of the query. The method may comprise providing the input to the generative machine learning model and in response receiving an output of the generative model.

The attributes may have corresponding values. That is to say, the plurality of attributes may form a feature vector of values, or otherwise be represented as a set of attribute, value pairs.

The optional features defined above in relation to the first aspect may be combined in any combination. Accordingly, each sentence in the optional features defined above can be read as if it is a dependent claim referring to the features of any preceding sentence.

According to a second aspect of the disclosure, there is provided a computer system comprising a processor and a memory storing instructions, the instructions when executed by the processor causing the system to carry out the method of the first aspect.

According to a third aspect of the disclosure, there is provided a computer-readable medium comprising instructions, which when executed by a processor cause the processor to carry out the method of the first aspect. The medium is suitably non-transitory and/or tangible.

According to a fourth aspect of the disclosure, there is provided a computer program product comprising instructions which when executed by a processor cause the processor to carry out the method of the first aspect.

Although at least some aspects of the embodiments described herein with reference to the drawings comprise computer processes performed in processing systems or processors, the invention also extends to computer programs, particularly computer programs on or in a carrier, adapted for putting the invention into practice. The program may be in the form of non-transitory source code, object code, a code intermediate source and object code such as in partially compiled form, or in any other non-transitory form suitable for use in the implementation of processes according to the invention. The carrier may be any entity or device capable of carrying the program. For example, the carrier may comprise a storage medium, such as a solid-state drive (SSD) or other semiconductor-based RAM; a ROM, for example a CD ROM or a semiconductor ROM; a magnetic recording medium, for example a floppy disk or hard disk; optical memory devices in general; etc.

The examples described herein are to be understood as illustrative examples of embodiments of the invention. Further embodiments and examples are envisaged. Any feature described in relation to any one example or embodiment may be used alone or in combination with other features. In addition, any feature described in relation to any one example or embodiment may also be used in combination with one or more features of any other of the examples or embodiments, or any combination of any other of the examples or embodiments. Furthermore, equivalents and modifications not described herein may also be employed within the scope of the invention, which is defined in the claims.

1. A computer-implemented method comprising:
receiving a plurality of input data items, each input data item of the plurality of input data items comprising:
a first plurality of attributes representative of characteristics of the input data item, the first plurality of attributes having a first plurality of dimensions,
a treatment variable associated with the input data item; and
an outcome variable representative of an outcome associated with the input data item;
generating, for each of the plurality of input data items, a second plurality of attributes from the first plurality of attributes, the second plurality of attributes having a second plurality of dimensions smaller than the first plurality of dimensions;
selecting a first input data item having a first value for the treatment variable;
determining, in respect of the first input data item, a matching second input data item based on a distance along a manifold between the second plurality of attributes of the first input data item and the second plurality of attributes of the second input data item, the second input data item having a second value for the treatment variable, and
estimating an effect of the treatment based the respective outcome variables of the first input data item and second input data item.

2. The method of claim 1, comprising:
selecting a plurality of further first input data items having the first value for the treatment variable;
determining respective matching further second input data items for each of the plurality of further first input data items, the respective further second input data items having the second value for the treatment variable;
estimating an average treatment effect based on the respective outcome variables of each further first input data item and its respective matched further second input data item.

3. The method of claim 1, wherein the mapping comprises:
determining, based on a training data set, a mapping for generating the second plurality of attributes from the first plurality of attributes;
applying the determined mapping to the plurality of attributes of the input data items.

4. The method of claim 3, wherein determining the mapping comprises determining a linear projection from the first plurality of attributes to the second plurality of attributes.

5. The method of claim 4, comprising fitting a parameterized Riemannian metric to an output of the linear projection.

6. The method of claim 3, wherein:
determining the mapping comprises training a non-linear dimensionality reduction model based on the training data set; and
applying the determined mapping comprises providing the first plurality of attributes to the non-linear dimensionality reduction model and in response receiving the second plurality of attributes.

7. The method of claim 1, wherein determining the matching second input data item comprises determining a nearest neighbour of the first input data item having the second value for the treatment variable.

8. The method of claim 1, wherein determining the matching second data item comprises:
determining a plurality of matching second input data items based on the distance along the manifold; and
generating a matched data item based on the plurality of proximate second input data items.

9. The method of claim 1, wherein the distance along the manifold is a geodesic curve.

10. The method of claim 1, wherein the attributes are representative of patient data, the treatment is a medical intervention, and the outcome variable is representative of a medical outcome of a patient.

11. The method of claim 1, wherein the attributes are representative of image data.

12. The method of claim 1, wherein the manifold is a Riemannian manifold.

13. The method of claim 1, wherein the attributes are representative of sensor data.

14. The method of claim 1, wherein the attributes are representative of plant products.

15. The method of claim 1, wherein the attributes are representative of industrial machinery.

16. A computer system comprising a processor and a memory storing instructions, the instructions when executed by the processor causing the system to carry out a method comprising:
receiving a plurality of input data items, each input data item of the plurality of input data items comprising:
a first plurality of attributes representative of characteristics of the input data item, the first plurality of attributes having a first plurality of dimensions;
a treatment variable associated with the input data item; and
an outcome variable representative of an outcome associated with the input data item
generating, for each of the plurality of input data items, a second plurality of attributes from the first plurality of attributes, the second plurality of attribute having a second plurality of dimensions smaller than the first plurality of dimensions, the representation space having a form of a manifold;
selecting a first input data item having a first value for the treatment variable;
determining, in respect of the first input data item, a matching second input data item based on a distance along a manifold between the second plurality of attributes of the first input data item and the second plurality of attributes of the second input data item, the second input data item having a second value for the treatment variable, and
estimating an effect of the treatment based the respective outcome variables of the first input data item and second input data item.

17. The system of claim 16, the instructions when executed by the processor further causing the system to carry out:
determining, based on a training data set, a mapping for generating the second plurality of attributes from the first plurality of attributes;
applying the determined mapping to the plurality of attributes of the input data items.

18. The system of claim 16, wherein the attributes are representative of patient data, the treatment is a medical intervention, and the outcome variable is representative of a medical outcome of a patient.

19. A non-transitory computer-readable medium comprising instructions, the instructions when executed by a processor causing the processor to carry out a method comprising:
receiving a plurality of input data items, each input data item of the plurality of input data items comprising:
a first plurality of attributes representative of characteristics of the input data item, the first plurality of attributes having a first plurality of dimensions,
a treatment variable associated with the input data item; and
an outcome variable representative of an outcome associated with the input data item;
generating, for each of the plurality of input data items, a second plurality of attributes from the first plurality of attributes, the second plurality of attribute having a second plurality of dimensions smaller than the first plurality of dimensions, the representation space having a form of a manifold;
selecting a first input data item having a first value for the treatment variable;
determining, in respect of the first input data item, a matching second input data item based on a distance along a manifold between the second plurality of attributes of the first input data item and the second plurality of attributes of the second input data item, the second input data item having a second value for the treatment variable, and
estimating an effect of the treatment based the respective outcome variables of the first input data item and second input data item.

20. The computer-readable medium of claim 19, the instructions when executed by the processor further causing the system to carry out:
determining, based on a training data set, a mapping for generating the second plurality of attributes from the first plurality of attributes;
applying the determined mapping to the plurality of attributes of the input data items.
